(19) United States
(12) Patent Application Publication
(10) Pub. No.: US 2014/0178910 A1
(43) Pub. Date: Jun. 26, 2014

(54) METHOD FOR ASCERTAINING THE ISCHEMIC LEVEL OF A PATIENT WITH SUSPECTED STROKE

(71) Applicants: Ernest Kapetanovic, Dusseldorf (DE); Samir Yastas, Meerbusch (DE); Wolfram Doehner, Schildow (DE)

(72) Inventors: Ernest Kapetanovic, Dusseldorf (DE); Samir Yastas, Meerbusch (DE); Wolfram Doehner, Schildow (DE)

(73) Assignee: DIAGENICS SE, Luxembourg (LU)

(21) Appl. No.: 14/126,260

(22) PCT Filed: Jun. 11, 2012
(86) PCT No.: PCT/EP2012/061012

§ 371 Date: Mar. 17, 2014

(30) Foreign Application Priority Data
Jun. 15, 2011 (DE) 10 2011 051 076.1

(51) Int. Cl. C12Q 1/48

(52) U.S. Cl. CPC C12Q 1/48
USPC 435/15; 435/194

(57) ABSTRACT

A method for determining the ischemic levels of suspected stroke patients comprises the following steps:
Taking a blood sample from the suspected stroke patients,
Determining the concentration of Glycogen Phosphorylase BB (GPBB) in this blood sample

### FIELD OF THE INVENTION

The invention relates to a method for determining the ischemic level of suspected stroke patients, as well as an appropriate biomarker and its corresponding use.

### DESCRIPTION OF PRIOR ART

From the WO 2003/046140 a method is known for the detection of heart disease, in particular of myocardial infarction, in which the presence is determined of at least two antigens in a blood sample. It is an early detectable antigen, namely, Glycogen Phosphorylase BB, and a late-elevated antigen, namely, Troponin-I.

According to WO 2008/064903, specific antibodies are used for the detection of native Glycogen Phosphorylase BB in human samples for the diagnosis of acute coronary syndromes, whereas these antibodies are derived from human Glycogen Phosphorylase enzymes BB.

Glycogen Phosphorylase (GP) is an enzyme that occurs in the form of three different isoenzymes in the body. Glycogen Phosphorylase Isoenzyme BB is one of them; it is found in large concentrations in the heart and brain. Glycogen Phosphorylase is generally a glycogen metabolizing allosteric enzyme. The access number of Glycogen Phosphorylase BB in NCB is DSM ACC 2834 and DSM ACC 2835.

The present invention relates, above all, to the so-called stroke (cerebral apoplexy). For example, atherosclerosis leads to a blockage of the arteries, which in turn can lead to CNS-related symptoms, including loss of language and/or modification of the language and also partial or total paralysis of certain areas of the body, including the facial muscles and/or other areas of the body, or similar symptoms. The therapeutic regimen includes an anticoagulant, and/or anti-platelet medications and thrombolytic agents and is critical for the diagnosis of ischemic stroke and/or a partial or full success of the revascularizations.

### THE OBJECTIVE

The objective of the present invention is to significantly improve the detection of stroke.

### REACHING THE OBJECTIVE

The process according to the invention contains the following steps that are used to determine the ischemic level of suspected stroke patients:
withdrawing a blood sample from the suspected stroke patients,
determination of the concentration of Glycogen Phosphorylase BB (GPBB) in the blood sample.

In a preferred embodiment, Glycogen Phosphorylase BB shows an epitope as described in the above-mentioned WO 2008/064903. Furthermore, Glycogen Phosphorylase BB is supposed to have a sequence as described in the international patent application.

The present invention also comprises a diagnostic biomarker for detecting ischemic levels of suspected stroke patients, characterized by determining the concentration of Glycogen Phosphorylase BB in the suspects' blood samples, whereas the determination takes place according to the process as described in particular in WO 2008/064903 or EP 1461616.

Another claim is the use for the determination of Glycogen Phosphorylase BB concentration in the blood of suspected stroke patients for determining a stroke.

### Statistical Analysis of GPBB Application in Patients with Acute Stroke

### 1. Description of the Statistic

| | |
|---|---|
| Patients with acute ischemic stroke: | N = 116 |
| Patients with TIA (transient ischemic attack), | N = 16 |
| Control subjects | N = 46* |

*Control subjects: Elderly subjects who have been admitted to A&E (Accident and Emergency) with symptoms similar to an acute cerebral event, which are not health related and are also not cerebral events attributable to stroke, but require an A&E admission.

values mean±SD (standard deviation)

| | | | | | stroke ./. |
|---|---|---|---|---|---|
| | control | | | | control |
| variable | subjects | patients with TIA | stroke patients | ANOVA | subjects |
| GPBB | 9.547 ± 7.407 | 12.898 ± 18.646 | 15.406 ± 16.702 | 0.08 | 0.027 |
| (ng/ml) | | | | | |
| NIHSS | 0 ± 0 | 1.6 ± 1.1 | 5.1 ± 3.0 | <0.0001 | <0.0001 |
| age (y) | 66.4 ± 16.7 | 67.9 ± 16.0 | 73.3 ± 11.1 | 0.007 | 0.003 |
| Glucose | 125 ± 48 | 113 ± 17 | 128 ± 32 | 0.4 | 0.7 |
| (mg/dl) | | | | | |
| HbA1c | 6.0 ± 1.2 | 6.2 ± 0.9 | 6.3 ± 1.0 | 0.3 | 0.15 |
| CrP (mg/dl) | 2.01 ± 4.88 | 1.07 ± 2.33 | 1.23 ± 2.04 | 0.30 | 0.14 |
| CK | 244.9 ± 763.9 | 96.0 ± 104.1 | 137.4 ± 193.2 | 0.23 | 0.15 |
| Cholesterol | 193.3 ± 71.0 | 196.9 ± 33.5 | 193.3 ± 43.2 | 0.96 | |
| Triglycerides | 115.3 ± 79.4 | 145.0 ± 58.1 | 123.6 ± 90.2 | 0.5 | 0.6 |
| GPT | 53.5 ± 98.4 | 27.5 ± 16.8 | 27.2 ± 19.4 | 0.17 | 0.005 |
| GOT | 149.0 ± 265.5 | 16.0 ± 1.4 | 32.2 ± 16.2 | 0.027 | 0.008 |
| Thrombocytes | 261 ± 86 | 268 ± 84 | 266 ± 68 | 0.9 | |
| Creatinin | 1.07 ± 0.2 | 1.21 ± 0.6 | 1.1 ± 0.4 | 0.5 | 0.7 |

### No Influence of Age:

The stroke patients were older in comparison with non-stroke patients.
There was no correlation found between age (independent) and GPBB (dependent variable): r: 0.018, p=0.8
Age is, therefore, not likely to affect the observed differences between the groups.

### 2. Grouping for Clinical, Biochemical Characteristics Based on GPBB

Between the stroke patients, clinical variables had no or only a slight influence on the GPBB concentration
IHD: ischemic heart disease
HTN: hypertension,
DM: diabetes
HLP: hyperlipoproteinemia
PEnk: P Enkephalin
only stroke patients

| Grouping variable | GPBB mean ± SD | GPBB mean ± SD | P |
|---|---|---|---|
| Gender male:female | 14.9 ± 15.4 | 16.0 ± 18.5 | 0.7 |
| IHD n = 72/y = 44 | 16.5 ± 16.9 | 16.7 ± 16.3 | 0.3 |
| HTN n = 23/y = 93 | 14.2 ± 15.9 | 20.4 ± 19.1 | 0.1 |
| DM n = 80/y = 36 | 13.7 ± 14.9 | 19.2 ± 19.8 | 0.10 |
| HLP n = 55/y = 61 | 13.5 ± 15.1 | 17.0 ± 17.9 | 0.2 |
| Smokers | 14.9 ± 16.9 | 17.1 ± 16.1 | 0.5 |
| n = 90/y = 26 | | | |
| Family history = | 14.7 ± 16.5 | 22.8 ± 17.8 | 0.14 |
| 148/y = 14 | | | |
| NIHSS 0-6/7-12 | 15.8 ± 17.7 | 14.4 ± 14.5 | 0.6 |
| PEnk </>median | 10.8 ± 11.3 | 19.4 ± 19.7 | 0.006 |
| PEnk 1/2 + 3 tertile | 13.4 ± 19.1 | 18.7 ± 19.1 | 0.10 |

3. Commonality and Correlation Analysis

GPPB correlated with P Enkephalin, with glucose and GOT (heart muscle damage or liver cell damage)

| Variable | R value | P value |
|---|---|---|
| P Enkephalin | 0.23 | 0.0125 |
| Glucose | 0.32 | 0.006 |
| HbA1c | 0.078 | 0.5 |
| GOT | 0.57 | 0.0002 |
| GPT | | ns |
| Age | | ns |
| NIHSS | | ns |
| CK | 0.2 | 0.036** |

**after exclusion of two outliers with acute MI.

No correlation was found to the lipids (CHOL, HDL, LDL, TG), thrombocytes, Ceratinin, CrP

### SUMMARY

1. A difference in average value of GPBB plasma levels of stroke and non-stroke (TIA and stroke-like) patients could be observed.
2. The GPBB plasma level in some typical patients with additional diseases including HTN, HLP, DM, are not affected (distorted).
3. The GPBB plasma level was not affected (disturbed) by a number of biochemical variables.
4. TIA patients have a lower GPBB value similar to non-stroke patients, however, there are some outliers (inexplicably) with very high values.
5. GPBB is related to P Enkephalin (blood-brain barrier markers) but also to glucose levels and GOT (indicator of heart muscle or substantial liver cell damage) and CK.

1. A method for determining ischemic levels of suspected stroke patients, comprising the steps of:
a. collecting a blood sample from the suspected stroke patients, b. determining the concentration of Glycogen Phosphorylase BB (GPBB) in said blood sample.

2. The method according to claim 1, characterized by Glycogen Phosphorylase BB having an epitope, as described in WO 2008/064903.

3. Method according to claim 2, characterized by Glycogen Phosphorylase BB having a sequence as described in WO 2008/064903.

4. Diagnostic biomarker for ascertaining the ischemic levels of a suspected stroke patients is characterized by a concentration-determination of Glycogen Phosphorylase BB (GPBB) in a blood sample taken from the suspected stroke patient, in which the determination is made by the process as described in particular in WO 2008/064903 or EP 1461616.

5. Biomarker according to claim 4, characterized by Glycogen Phosphorylase BB having an epitope, as described in WO 2008/064903.

6. Biomarker according to claim 5, characterized by Glycogen Phosphorylase BB having a sequence as it is described in WO 2008/064903.

7. Use of determination of the concentration of Glycogen Phosphorylase BB in the blood of a suspected stroke patient to identify a stroke.

8. Use of a kit for determination of the concentration of Glycogen Phosphorylase BB in the blood of a suspected stroke patient.
